(19) United States
(12) Patent Application Publication
(10) Pub. No.: US 2021/0007810 A1
(43) Pub. Date: Jan. 14, 2021

(54) MEDICAL IMAGING SYSTEM AND METHOD FOR DETECTING THE POSITION THEREOF

(71) Applicant: 270 SURGICAL LTD., Netanya (IL)

(72) Inventor: David RAINIS, Haifa (IL)

(21) Appl. No.: 16/977,483

(22) PCT Filed: Mar. 25, 2019
(86) PCT No.: PCT/IL2019/050333

§ 371 Date: Sep. 2, 2020

Related U.S. Application Data
(60) Provisional application No. 62/649,609, filed on Mar. 29, 2018.

(51) Int. Cl. A61B 34/20; A61B 1/00; A61B 1/04; G16H 30/40

(52) U.S. Cl. CPC A61B 34/20; A61B 1/00009; A61B 1/04; A61B 1/313; A61B 1/00078; G16H 30/40; A61B 2034/2065; A61B 1/00006

(57) ABSTRACT

The present invention discloses a method and a system for managing optical gear module in conjunction with a medical imaging device, according to a predefined set of conditional events. The medical imaging device disclosed herein can comprise two section members directly connected to a rigid scope. Said two section members may be an elongated rigid shaft tube and a distal tip. The distal tip can comprise an optical gear module required for some medical procedures. In some cases, at least one optical gear modules are located in the distal tip, each optical gear module can comprise a camera comprises a lens assembly and a sensor and light sources required for the camera functioning. The disclosed system can detect video signal/s captured by at least one of the cameras associated with the at least one of the optical gear modules. In some cases, upon detecting a video signal, the system may identify if the rigid scope position either outside or inside the patient body. The system can also be configured initiating operational events according to a predefined conditional event set, in case the rigid scope position is detected to be within the patient body. The system may comprise a controller for analyzing an intensity level of colors received in a colored light received at the medical imaging endoscope.

### FIELD OF THE INVENTION

The present invention generally relates to the field of medical instruments designed to capture images from inside the patient's body.

### BACKGROUND OF THE INVENTION

Endoscopes have been utilized to perform operations in the internal organs of the body through small incision in skin. Over the years, numerous rigid and semi rigid endoscopes have been developed and categorized according to specific applications, such as laparoscopes arthroscopes, cystoscopes, ureteroscopes and others. In multiple cases, such operations require the aid of a camera. Furthermore, in some cases, procedures which involve inspection of a region inside confined area or a specific body cavity or organ, may be required to involve more than one camera. A laparoscope is a medical imaging device utilized to perform operations in the abdomen or pelvis through small incisions with the aid of a camera. It can either be used to inspect and diagnose a condition or to perform surgery.

In some medical procedures, the used laparoscope may comprise more than one camera located at the front of the laparoscope. In some cases, optical components and electrical components may require recording the view captured by the cameras. In such cases, the medical imaging device may be connected to a system capable of receiving the video content transmitted from the camera and present it on a display. In some cases, it is required to record and save the recorded material transmitted by the medical imaging device. The recorded video content may be utilized later on to analysis the medical procedure and the patient status.

### SUMMARY OF THE INVENTION

The present invention discloses a method and a system for managing optical gear module in conjunction with a medical imaging device, according to a predefined set of conditional events. The medical imaging device disclosed herein can comprise two section members directly connected to a rigid scope. Said two section members may be an elongated rigid shaft tube and a distal tip. The distal tip can comprise an optical gear module required for some medical procedures. In some cases, at least one optical gear modules are located in the distal tip, each optical gear module can comprise a camera comprises a lens assembly and a sensor and light sources required for the camera functioning.

The system disclosed in the present invention which manages the optical gear module/s can be configured such that, the system can detect video signal/s captured by at least one of the cameras associated with the at least one of the optical gear modules. In some cases, upon detecting a video signal, the system may identify if the rigid scope position either outside or inside the patient body. The system can also be configured initiating operational events according to a predefined conditional event set, in case the rigid scope position is detected to be within the patient body. The system may comprise a controller for analyzing an intensity level of colors received in a colored light received at the medical imaging endoscope. Such a controller can compare the intensity levels of colors to specific thresholds determine the position of said medical imaging endoscope. The controller may be configured to detect the video signal resulted by an image captured by the optical gear of the medical imaging endoscope device, and in case the video signal is detected to receive the color values of the RGB representing the intensity levels of RGB colors of the color space of the video image. The controller can also conduct a comparison process designed to compare the received color values of the RGB color with at least one predefined threshold. In some cases, the results of the comparison process may indicate that the distal tip of the medical imaging endoscope device position is within a body of a patient. The controller may be further configured to initiate operational events according to a predefined conditional event set, in case the rigid scope position is detected to be within the body of the patient.

The system disclosed in the present invention may utilize a method for identifying the intensity level of colors received in colored light received per second at the medical imaging device and according to specific thresholds determine the position of the rigid scope. For example, the system can analyze the intensity level of the red-light relative to the intensity level of other colors, and in case the red-light intensity level meets a specific level or specific threshold, the system may identify that the medical imaging endoscope is within the body and as a result, initiating operational events according to a predefined conditional event set. In some cases, such an action may be start or stop recording the digital video received by the camera/s, start or stop LEDs activity in the distal tip, start or stop insufflation process, start a medical procedure, stop a medical procedure, and the like.

In some embodiments of the present invention the medical imaging device may comprise a rigid elongated member and a distal tip which can be connected to the rigid section in a rigid manner. The distal tip may comprise a front camera located on a front planar surface of the distal tip and a second side camera located on a first lateral surface of the distal tip. In some embodiments of the present invention the system may analyze the position of the distal tip in accordance with a specific camera. For example, the system may detect that the intensity of the red-light reaches a specific level in a specific camera and as a result of that, cause to the LEDs associated with a specific camera to start illumination activities.

In some embodiments of the disclosed subject matter, the system may detect the distal tip position within a body by providing an input through the input device, connected to the system. An input device such as a handle or an external keyboard or touchscreen display. A user utilizing the input device may also be enabled to update the system on the rigid scope position. For example, a user utilizing the input device may press a key or a key combination on the keyboard, to inform the system that the rigid scope position is in the human body.

In some cases, the medical imaging endoscope may comprise a handle which can be utilized for guiding elongated rigid shaft within a body cavity. The handle may comprise one or more buttons and/or switches which control functions such as an input device.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A demonstrates a medical imaging device comprising an optical gear, according to exemplary embodiments of the disclosed subject matter;

FIG. 1B demonstrates a medical imaging device comprising a rigid shaft which comprises an optical gear required for the operation of the medical imaging device, according to FIG. 1A;

FIG. 2A illustrates a medical imaging system, according to some embodiments;

FIG. 2B is a block diagram illustrating the medical imaging endoscopy system in which the method for detecting the position of a rigid shaft of the medical imaging endoscope provided by the present specification may be implemented;

FIG. 3A is a flowchart illustrating a process which triggers an event of a medical imaging device, in accordance with exemplary embodiments of the present invention;

FIG. 3B is a flowchart illustrating a process conducted by a controller which h triggers an event of a medical imaging device, in accordance with exemplary embodiments of the present invention;

FIG. 4A is a flowchart illustrating a process operable by a medical imaging system for identifying an endoscope position, according to exemplary embodiments of the present invention; and

FIG. 4B is a flowchart illustrating the steps performed by the process pipeline shown in FIG. 4A.

### DETAILED DESCRIPTION OF THE INVENTION

The subject matter in the present invention discloses a medical imaging device comprising an imaging rigid endoscope designed to aid medical procedures such as inspection or surgery procedures in the abdomen or pelvis through small incisions. Such medical imaging device can comprise one or more cameras designed to aid medical procedures such as inspection or surgery.

As used in the specification, the term “optical gear module” or “optical gear” is used to depict a set of components that allows the medical imaging device to capture light and transform that light into at least one image. In some embodiments, lenses are employed to capture light and image capturing devices, such as sensors, are employed to transform that light into at least one image. In some embodiments, a camera comprises a plurality of optics such as lens assembly and sensor, can be configured to receive reflected light from target objects. In some embodiments, an optical gear located in the distal tip can comprise sensor, lenses (e.g., camera) and light sources required for the sensor functioning.

Image capturing devices may be Charged Coupled Devices (CCD's) or Complementary Metal Oxide Semiconductor (CMOS) image sensors, or other suitable devices having a light sensitive surface usable for capturing an image. In some embodiments, a sensor such as a Charge Coupled Device (CCD) or a Complementary Metal Oxide Semiconductor (CMOS) image sensor (for detecting the reflected light received by an optical element), is employed.

It should also be noted that a plurality of terms, as follows, appearing in this specification are used interchangeably to apply or refer to similar components and should in no way be construed as limiting: A “medical imaging endoscope device” may also be referred to as “rigid scope” or “medical imaging endoscope”. A “camera” may also be referred to as an image capturing device/component, comprises a lens assembly and sensor. An “illuminator” may also be referred to as an “illumination source”, and in some embodiments, an LED.

It is noted that the term “endoscope” as mentioned to herein may refer particularly to a rigid scope and semi-rigid scope such as laparoscope, according to some embodiments of the disclosed subject matter, but is not limited only to laparoscope, and may include other applications such as industrial applications. The term “endoscope” may refer to any instrument used to examine the interior of a hollow organ or cavity of the body.

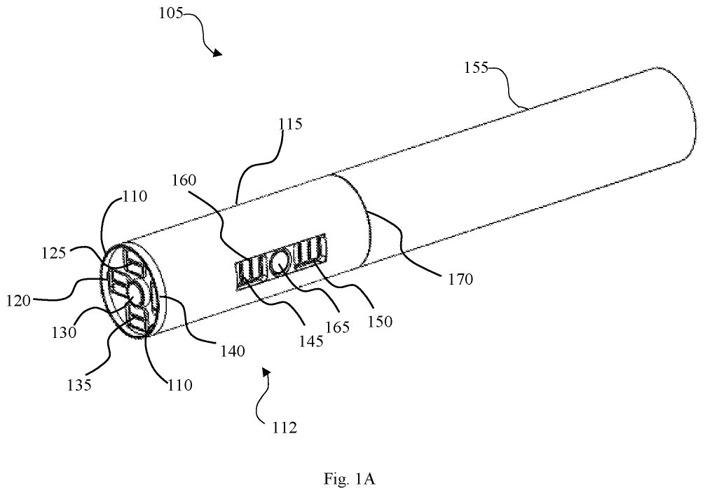

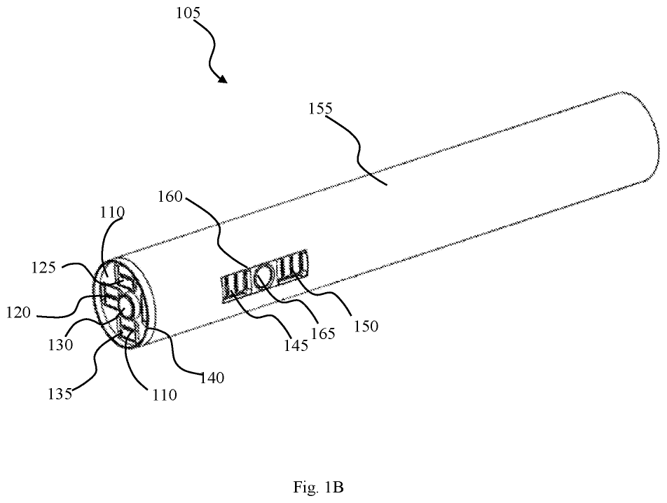

FIG. 1A demonstrates a medical imaging device comprising at least one camera, according to exemplary embodiments of the disclosed subject matter. FIG. 1 shows a rigid scope 105 comprising a rigid shaft 155 designed to be directly connected to a distal tip component 115, wherein distal tip component 115 may cover the optical gear (not shown) of the distal tip 112. In such cases, the seamline 170 outlines the connection line between the elongated rigid shaft 155 and the distal tip component 115. In some cases, the elongated rigid shaft 155 and the distal tip component 115 may be connected by an adhesive material that seals the connection at the seamline 170. In some other cases, the elongated rigid shaft 155 and the distal tip component 115 may be connected by soldering. In possible embodiments of the disclosed subject matter, the elongated rigid shaft 155 and the distal tip component 115 may be connected by a screwing mechanism which fastens the elongated rigid shaft 155 and the distal tip component 115 together. In yet another embodiment (not shown), the elongated rigid shaft 155 extends along the length of rigid scope 105 covering a distal tip 112.

The distal tip 112 may function as a multi-camera section member designed to house at least one camera. In some cases, the cameras of the distal tip 112 may be located in the front at the planar surface 110. Additional cameras may be located at the lateral round surface of the distal tip component 115. The distal tip component 115 may also comprise an aperture 160 shaped to house a second side camera 165 and provide the opening required for the field of view of the second side camera 165. In some cases, the aperture 160 may be covered by a transparent layer, such as glass or plastic, to isolate the side camera 165 from the patient's tissue. In some other cases, aperture 160 may be covered by an optical window or more than one optical window.

In some embodiments, the distal tip 112 may comprise a first side camera (not shown) located at the opposite side of the distal tip 112. The aperture 160 also enables emission of light from side illuminators 150, and 145 which provide the light source of the side camera 165. In some cases, the light may be emitted by dedicated section illuminators such as light-emitting diodes, also known as LED.

The distal tip 112 may also comprise a front camera 130 situated at the center of a planar surface 110 which can house the front camera 130 and provide the opening required for the field of view of the front camera 130. The planar surface 110 also comprise front illuminators 120, 125, 135, and 140 which provide the required source of light for front camera 130. In other possible embodiments, the number and location of front illuminators may vary for example, less than 4 set of illuminators or more wherein each set of illuminators has 1, 2, 3, 4 or more LED and may emit the same light spectrum or different light spectrum.

In some other cases, a portion of the illuminators 150, 145, 120, 125, 135, and 140 which provide the required source of light of the rigid scope 105 may be at different colors at the visible light. For example, the light source of the rigid scope 105, may comprise LED's emitting a white light LED and or other colors such as blue, red, yellow, green, or any combination thereof. In some cases, the light emitted by the LED's may be at the spectrum of the non-visible light. For example, a source light can provide a light at the infrared spectrum, ultra-violate, x-ray, and the like

FIG. 1B demonstrates a medical imaging device comprising a rigid shaft which comprises an optical gear module required for the operation of the medical imaging device, according to FIG. 1A. FIG. 1B shows a rigid scope 105 comprising one rigid shaft 155 without a distal tip. The rigid shaft 155 may function as an optical gear section member designed to house at least one optical gear. In some cases, the optical gear/s may be positioned at the edge of the rigid shaft 155 located in the front at the planar surface 110. In some other cases, the optical gear/s may be located at the cylindrical round surface of the rigid shaft 155 at a distance between 6.5 to 40 millimeters from front panel 110. In yet another embodiment, a front camera may be positioned at the edge of the rigid scope at front panel 110 and one or more side cameras may be located at the cylindrical round surface of the rigid shaft 155 at a distance between 6.5 to 40 millimeters from front panel 110.

The rigid shaft 155 may also comprise an aperture 160 shaped to house a second side camera 165 and provide the field of view operatively required for the second side camera 165. In some embodiments of the disclosed subject matter, the rigid shaft 155 may comprise a first side camera (not shown) located at the opposite side of the rigid shaft 155 within a first side aperture (not shown). The aperture 160 also houses side illumination modules 150 and 145 which provide the light source of the side camera 165 and configured to provide the required illumination activities required by the side camera 165. In some cases, the light source may be emitted by dedicated illuminators such as light-emitting diode, also known as LED. In some cases, each illumination module has 1, 2, 3, 4 or more LED and emit different light spectrum as will explained above.

The rigid shaft 155 may also comprise a front camera 130. In an embodiment, front camera 130 may be situated closer to one of at the center of a front planar surface 110 which can house the front camera 130 and provide the field of view operatively required for front camera 130. The front planar surface 110 may also comprise front illuminators' sets 120, 125, 135, and 140 which provide the required source of light for front camera 130. In some cases, each illumination module has 1, 2, 3, 4 or more LED and may emit the same light spectrum or different light spectrum as was explained above. In another embodiment, front camera 130 may be situated at the center of the front planar surface 110.

In some cases, the rigid shaft 155 may be prepared as a one-piece. For example, rigid shaft 155 may be prepared by a molding process. In some cases, the preparation process of the rigid shaft 155 may also comprise a milling process for creating the apertures for the cameras, the rounded surfaces, the planar surfaces, the room for the cameras, and the like.

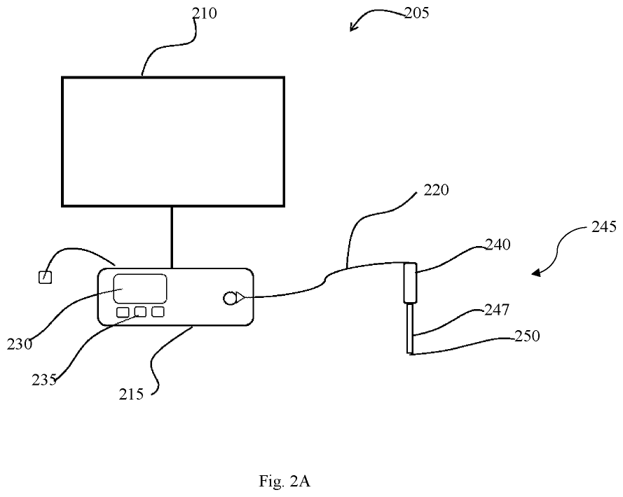

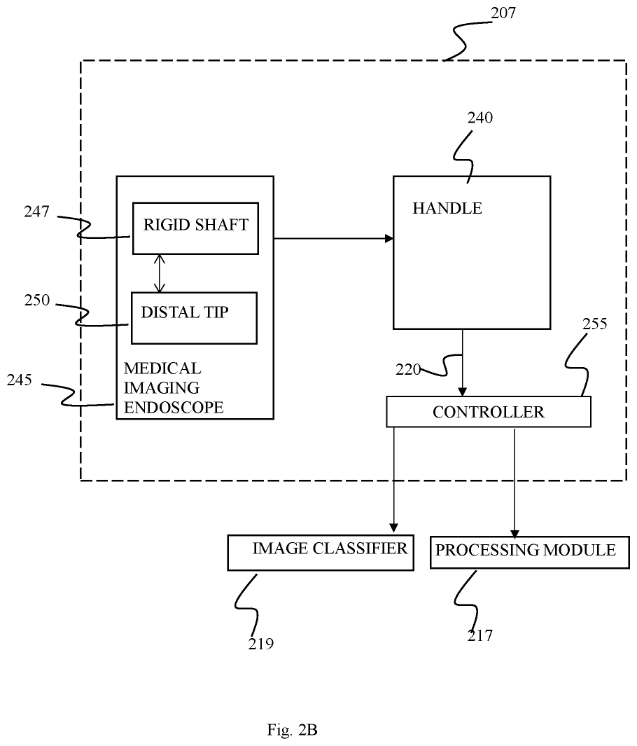

FIG. 2A shows a medical imaging system, according to exemplary embodiments of the disclosed subject matter. Medical imaging system 205 may comprise a display unit 210, and a main control unit 215 designed to be connected to a medical imaging endoscope 245 by a utility cable 220. The medical imaging endoscope 245, shown in this exemplary embodiment, can be a camera elongated rigid shaft such as an endoscope, which may comprise a handle 240, to which a rigid shaft 247 may connect in a detachable manner or as one unit. The rigid shaft 247 terminates with a distal tip section 250. Handle 240 may be utilized for guiding rigid shaft 247 within a body cavity. In some cases, the handle 240 may comprise one or more buttons and/or switches (not shown) which control functions as aforementioned as well as zoom, focus, record and the like. Distal tip section 250 may comprise at least one optical gear module providing the user with a wider field of view necessary for examine the region of interest as well as medical instruments and other organs surround the treated area.

The utility cable 220, may connect between handle 240 and the main control unit 215. The utility cable 220 may comprise therein one or more electrical channels. The electrical channel(s) may comprise at least one data cable for receiving video signals from the at least one optical gear module of the distal tip. In an embodiment, the electrical channel(s) may comprise at least one data cable for receiving video signals from a front and at least one side cameras, as well as at least one power cable for providing electrical power to the cameras and to the illuminators associated with the at least one optical gear module. In another embodiment of the present invention, wireless communication between handle and main control unit can be configured and utilized.

The main control unit 215 comprises the controls, such as controls 235, required for displaying the images of internal organs captured by the optical gear module/s of distal tip section 250. The main control unit 215 can direct power transmission to the endoscope's distal tip 250 components, such as for the camera/s and illuminators. One or more input devices, such as a keyboard, a mouse, a touch screen, and the like may be connected to the main control unit 215 for the purpose of controlling thereof. In the exemplary embodiment shown in FIG. 2, the main control unit 215 comprises a display 230 for displaying operational information concerning the operation and activities of the medical imaging endoscope 245. The operational information can be changed and/or determined by operation buttons which can also be located on the main control unit 215. The images/videos can be displayed separately on one or more display units, such as display unit 210 by uploading information from the main control unit 215, either side-by-side or interchangeably, namely, the user operate the main control unit 215 can switch between views from the different viewing elements manually. Alternatively, these video streams can be processed by the main control unit 215 to combine them into a stitched image based on an overlap between fields of view of the cameras.

The main control unit 215 may also be designed to receive the digital inputs from the distal tip 250 of medical imaging endoscope 245 and convert said digital input to a video stream/s displayed on the display unit 210. The display unit 210 can further be operative to display a user interface for allowing an operator to set various features of the endoscopy system. In some cases, the main control unit 215 can also comprise a controller programmed to interlock with the camera rigid endoscope and configured to control some operational tasks of the medical imaging 245. The operational tasks can such as: Start /stop recording the field captured by the optical gear module/s, start/stop insufflation, start/stop LEDs activity, report the state of the optical gear module/s, to the to an external system, start a medical procedure, stop a medical procedure, and the like. Thus, the controller can also comprise a software unit operable on a processor and designed to execute the instructions stored in the computer-readable storage medium. In some cases, the controller can also comprise an independent base board module. In some cases, such an independent base board module can be a Field Programmable Gate Array (FPGA), or in some cases an Application Specific Integrated Circuit (ASICS), configured to implement an algorithm to detect changes in Red Grin Blue (RGB) image colors obtained by the plurality of optical gear modules. In some cases, the algorithm detecting changes in RGB color can be configured to detect if the imaging rigid shaft 247 is positioned outside or inside a patient body.

In some embodiments of the present invention, the controller may be just a software unit operable on a processor and configured to interlock with the camera rigid endoscope. For example, the control unit 215 can be implemented with a processor and the controller can be operable on the processor of the control unit.

In some cases, the main control unit 215 may also be configured to execute the operational tasks, according to a predefined set of conditional events associated with the rigid shaft 247 positions. For example, the main control unit 215 can start recording the field captured by one of the optical gears, in case a multi camera rigid endoscope position is detected to be within the patient body. Another exemplary case can be such that the insufflation process is triggered according to a predefined control system adapted to identify gas, such as smoke, in the camera field of view and/or pressure change by a pressure sensor configured to detect pressure external to the medical imaging device and internal to the body cavity. In such embodiments, the control system automatically evacuates gas from the body cavity. PCT application Ser. No. PCT/IL2017/051226, which relates to the Application of the present specification, entitled “A Medical Device Having a Gas Path Apparatus” and filed on Nov. 12, 2017 which, in turn, relies upon U.S. Provisional Patent Application No. 62/427825, filed on Nov. 30, 2016, for priority, is one example of an insufflation process and is herein incorporated by reference in its entirety. In some cases, the operational tasks may be controlled by the main control unit 215, according to the change detection results as received from the controller. In some other cases, the operational tasks may be controlled by the main control unit 215, according to a computerized input indicating the position of the rigid shaft 247 of medical imaging endoscope 245. For example, the main control unit 215 can receive a computerized input received from an input device and indicating the position of the medical imaging endoscope 245, wherein the computerized input indicates that the rigid shaft 247 is within a patient body. In such an exemplary case, the main control unit 215 may perform the operational tasks as define above, according to a predefined set of conditional events.

In some embodiments of the present invention, the controller can be located at handle 240. In such embodiments of the specification, the controller may comprise a processor and a computer-readable storage medium for executing the instruction. The controller located at handle 240 can also be configured to communicate with system main control unit 215.

In some cases, the main control unit 215 cam further comprise an image processing module (not shown) which can conduct a synchronized transferring of one or more video signals from the one or more cameras of the medical imaging endoscope 245 to the display unit 210. The image processing module can also be programmed to control a record of the at least one of optical gear module's video frame/image and or a video signal. In some embodiments, medical imaging system 205 further connects to an external system. The external system can be a hospital reporting system.

FIG. 2B is a block diagram illustrating a possible architecture of the medical imaging system in which the method for detecting the position of a rigid shaft of a medical imaging endoscope provided by the present specification can be implemented, according to FIG. 2A. FIG. 2B illustrates a possible architecture 207 which can comprise, a medical imaging endoscope 245 with a handle 240, which can detachably connect to, or built into a rigid shaft 247, and a controller 255. Further, rigid shaft 247 includes distal tip 250 comprising at least one optical gear. In some cases, a utility cable 220 is utilized to connect handle 240 with controller 255. In another embodiment of the disclosed subject matter the handle 240 can be connected to controller 255 via wireless communication. The controller 255 can be programmed to interlock with the medical imaging endoscope and configured to control some operational tasks of the medical imaging endoscope, as aforementioned. In some cases, the controller further configured to initiate an operational event, or operational events as aforementioned. In some cases, the controller is configured to initiate the operational event, or operational events once colors received in colored light meets specific thresholds as aforementioned.

In some embodiments of the disclosed subject matter, the controller 255 can be located at a control unit (such as main control unit 215 of FIG. 2A) as aforementioned. In some other cases, the controller 255 can be embedded into the handle 240. The controller 255 can be configured to connect to a processing module 217 and image classifier 219 adapted to detect and manipulate histograms, such as RGB color values, or YCbCr color values (where Y is luminance, Cb is blue-difference chroma, and Cr is red-difference chroma) and the like, received form image processing module 217. The histogram is defined as a numeric value representing the pixel sum of a specific color in the image frame of the video signal/s captured by at least one of the cameras. In some cases, the image classifier 219 can also adapt to define parameters such as received color values and received intensity levels of each image color within the received video frame/image and or a video signal. The intensity level of a color is the percentage the pixel sum of a specific color divided by the total sum of all colors. For example, the system can utilize a color range of Red, Blue, and Green, or in some cases, YCbCr color values to measure the intensity levels thereof in the image frame.

In some cases, the image classifier 219 can be a computerized process and/or a computerized set of instructions conducted by a process operable by the control unit (such as main control unit 215 of FIG. 2A). In some other cases, the image classifier 219 can be a computerized process and/or a computerized set of instructions conducted by a process operable by an external device (not shown). In some cases, the processing module 217 and the image classifier 219 can be located at a control unit (such as main control unit 215 of FIG. 2A) as aforementioned. In some other cases, the processing module 217 and the image classifier 219 can be lactated externally to the medical imaging system. The external device can be computerized device operated by a computerized instruction set. The device can be such as a personal computer, a tablet personal computer, a computer, a computerized telephone device and the like.

Controller 255 also interlocks with an image processing module 217 which enables synchronized transfer of one or more video signals from the one or more cameras of the medical imaging endoscope 245 to a display unit 210. Image processing module 217 can also be programmed to control a record of the at least one of optical gear module's video frame/image and or a video signal.

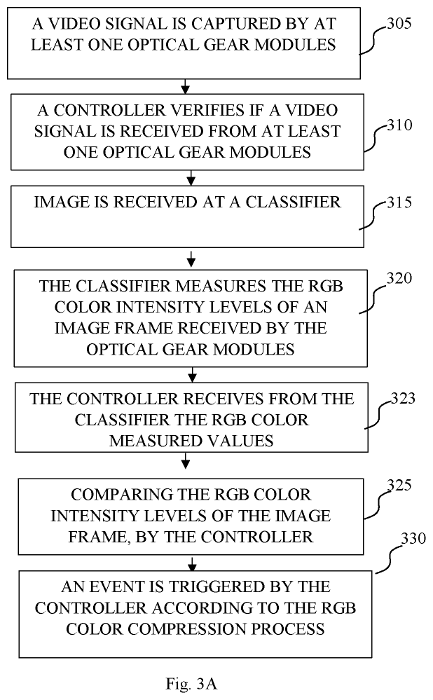

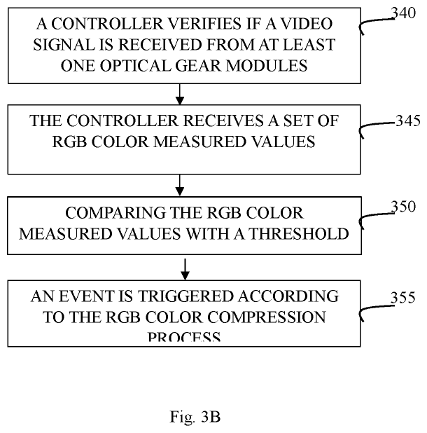

FIG. 3A is a flowchart illustrating a process which triggers an event of a medical imaging system, in accordance with exemplary embodiments of the present invention. At step 305 a video frame/image and or a video signal is captured by at least one optical gear module located within a distal tip of a medical imaging endoscope device. The frame/image and or video signal exits and transmitted from the distal tip into a main control unit of the medical imaging system as described in FIG. 2B. Such frame/image and or video signal can comprise objects and/or view captured by a camera located at the distal tip of medical imaging device.

At step 310 a controller, located within the main control unit or at the handle of the medical imaging endoscope device, verifies if the frame/image and or video signal is received from at least one optical gear modules. In some cases, the controller can be configured to probe the optical gear modules of the medical imaging endoscope device. For example, the controller can probe periodically the camera/s in a medical imaging endoscope device and verify if the frame/image and or video image can be detected. In some embodiments of the present invention, the optical gear module/s can be configured to trigger the controller to start operating in case at least one of the cameras captures objects, or any field of view.

At step 315 the frame/image and or video signal can be received by an image classifier wherein the digital video frames can be sent from the controller. At step 320 the classifier measures the intensity level of the RGB image frame colors received from the optical gear modules. In some cases, the medical imaging system can be designed to measure the intensity levels of the RGB color by the main control unit, as aforementioned. At step 323 the controller receives the RGB color measured values from the controller. In some cases, the RGB color value or color values can be the values measured by the classifier. In some embodiment of the disclosed subject matter, the classifier can be configured to measure the histograms of the colors of the image frame received from at least one optical gear modules located in the distal tip.

At step 325 the measured RGB color values can be compared by the controller with predefined thresholds. In such cases, the result of the comparison process conducted by the controller can be utilized to detect the distal tip of the medical imaging endoscope device position. Thus, the controller can be configured to compare the received color values with at least one predefined threshold. For example, the measured RGB color intensity level can exceed at least one threshold predefined in an RGB color range and thereby the controller, or in some cases the system, can determine whether the distal tip of the medical imaging endoscope device is positioned outside of a patient body, or inside the patient body. In some cases, the controller can be configured to receive additional parameters for the position detecting process. For example, a user operating the medical imaging endoscope device can set the controller such that diverse medical procedures may utilize different ranges of RGB color intensity level. Thus, the controller can identify that the distal tip of the medical imaging endoscope device is positioned outside the patient body, according to a specific predefined range of RGB color intensity levels, in case of a specific medical procedure. Yet, the controller can identify that the medical imaging endoscope device is positioned inside the patient body with the same specific predefined range of RGB color intensity level, in case of a different medical procedure.

At step 330 an event is triggered by the controller according to the results of the RGB color comparison process, as aforementioned. For example, in case the distal tip of the medical imaging endoscope device is identified as positioned inside the patient body, according to the results of the RGB color comparison process, the controller can send a command to start recording the video captured by at least one of the cameras of the optical gear. In some cases, the command can be sent to an image processing module which can receive the video signals and store the image frames of the video sequence in case a recording command was send by the controller.

FIG. 3B is a flowchart illustrating a process conducted by a controller which triggers an event of a medical imaging system, in accordance with exemplary embodiments of the present invention. At step 340 a controller, located within a main control unit or at a handle of a medical imaging endoscope device of the medical imaging system, verifies if the frame/image and or video signal is received from at least one optical gear modules. The controller can be programmed to interlock with the medical imaging endoscope device and configured to control some operational tasks of the medical imaging endoscope device. In some cases, the controller can be configured to probe the optical gear module/s of the medical imaging endoscope device. In some embodiments of the disclosed subject matter, the controller can be configured to trigger an external image classifier to measure the intensity levels of the RGB color received by the optical gear module/s of the medical imaging endoscope device. In some cases, such external image classifier is programmed to interlock with the medical imaging endoscope. Such external image classifier can be located on a computerized device such as a personal computer, a personal computerized mobile device, s Field Programmable Gate Array (FPGA), or in some cases an Application Specific Integrated Circuit (ASICS). In some cases, the external image classifier can be integrated with the control unit as aforementioned.

At step 345 the controller can receive the RGB color measured values. In some cases, the RGB measured values can be the values measured by an image classifier. In some embodiment of the disclosed subject matter, the image classifier can be configured to measure the colors' histogram of the image frame received from at least one optical gear modules located in the distal tip. At step 350 the measured RGB color values can be compared by the controller with predefined thresholds. At step 355 an event is triggered by the controller according to the results of the RGB color comparison process, wherein the controller compares the RGB color values with a predefined threshold, as aforementioned. For example, in case the distal tip of the medical imaging endoscope is identified as positioned inside the patient body, according to the results of the RGB color comparison process, the controller image classifier can trigger the controller, for example by sending a command to start recording the video captured by at least one of the cameras located at the distal tip. In some embodiments of the present invention, the usage of RGB color range can be replaced with YCbCr color values or other scales which can be utilized by a person having ordinary skills in the art to measure the intensity value of the color.

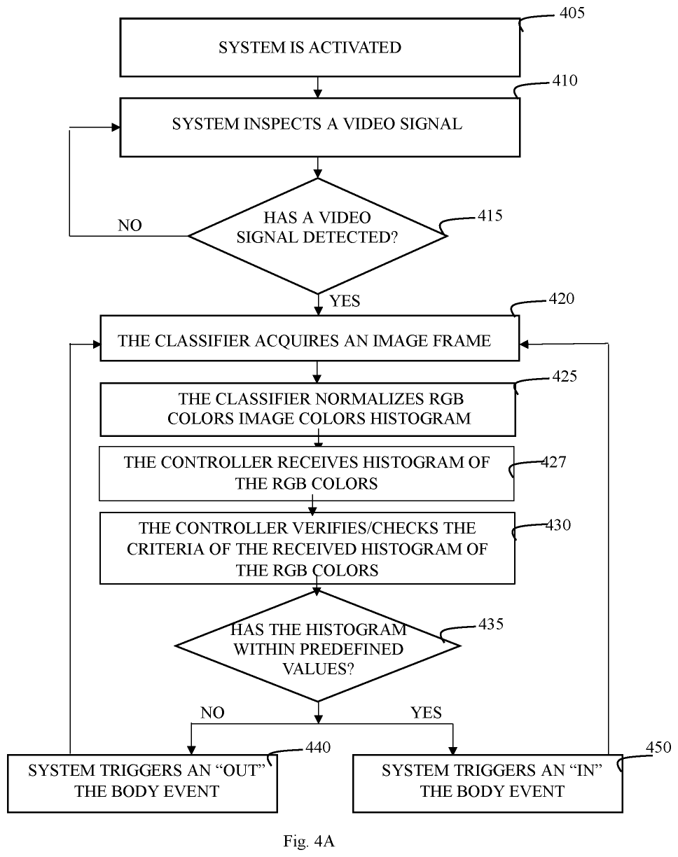

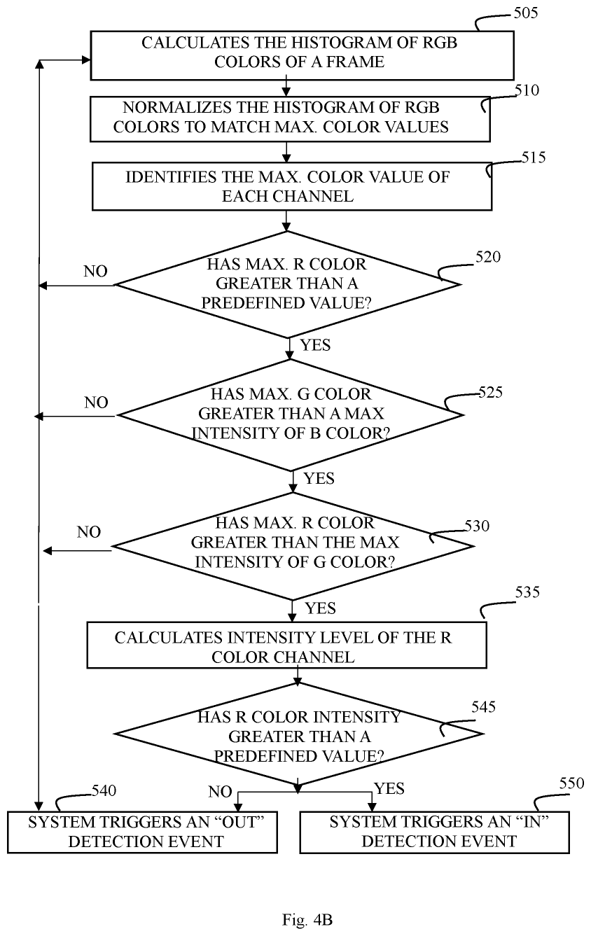

FIG. 4A is a flow chart showing the steps of an exemplary identification process operable by a medical imaging system for identifying a distal tip of a medical imaging endoscope position and controlling some operational tasks of the medical imaging system, according to exemplary embodiments of the present invention. At step 405 a system, wherein some cases can be the main control unit (for example, the main control unit 215 as shown in FIG. 2A), is activated. In some cases, in this activation step, the system can be charged up with electrical power, and a user can be able to operate the medical imaging system associated thereof. At step 410 a video signal from at least one optical gear module located on the distal tip (for example, the distal tip section 250 as shown in FIG. 2A) of the medical imaging endoscope (for example, the medical imaging endoscope 245 as shown in FIG. 2A) can be inspected by the system.

Furthermore, at step 410, if a video signal can be inspected the system then, at step 415 it is determined, by the system, if the multi camera medical imaging system detects a video signal in one of the at least one optical gear modules. In case the video signal module is not detected a process operated by the system returns the system to be set to the conditions of step 410. In some cases, the system can be configured to pause and verify periodically if video signal was detected by at least one of the optical gear modules. It should be noted that the operation of the system refers to a video signal as well as to video signals. In case video signal was detected by the system, the system can progress to step 420 in order to initiate an image classifier operation. For example, a camera can start capturing objects located at the camera's field of view and the electrical signals resultant of the video capturing can be transmitted to the system and interpreted as video signals. In such cases, the system can initiate the image classifier operation.

At step 420 the image classifier designed to measure the distribution of colors in an image acquires the image frame received form the digital video camera. Such image classifier can be a computerized process, or a computerized set of instructions conducted by a process as aforementioned. In some embodiments of the disclosed subject matter, the image classifier can receive the image frame of the video camera from the system which operates the classifier. In such configurations, the system can receive the video signal/s from at least one of the video cameras and then route the video signals to the classifier. In yet other embodiments of the subject matter the classifier can be configured to directly connect to at least one of the video cameras and receive the video signals.

In possible embodiments of the disclosed subject matter, a controller can perform the system tasks as described in steps 410, 415, and 420. Thus, the controller can inspect the video signal/s in the camera/s, and in case a video signal is detected, the controller can send the video signal to the classifier. In some cases, wherein a video signal is detected, the controller can send a command to the classifier to start receiving video signals from at least one of the cameras.

In some cases, the image classifier can utilize color values and intensity levels of the colors in the ranges of the image's color space. Color space can an abstract model which simply describes the range of colors, as values or color components (such as RGB color, YCbCr color (where Y is luminance, Cb is blue-difference chroma, and Cr is red-difference chroma), and the like). For example, the system can utilize a color space of Red, Green, and Blue, measure the intensity levels thereof in the image frame, and present the intensity levels of the Red, Green, and Blue in by a range of three (3) values.

At step 425 the classifier normalizes the values of histogram, according to the color range utilized by the classifier. For example, the classifier can divide each intensity level by the sum of all of the intensity levels. The sum of all of the intensity levels denoted S, wherein the intensity level of the red color denoted R, the intensity level of the green color denoted G, and the intensity level of the blue color denoted B, may be S=R+G+B. In such a case, normalizing the intensity levels may yield a normalized red intensity level as

$$\frac{R}{S} ,$$

a normalized green intensity level as

$$\frac{G}{S} ,$$

and a normalized blue intensity level as

$$\frac{B}{S} .$$

At step 427 the controller receives the histogram of the RGB colors, e.g., the normalized RGB color intensity levels, from the classifier, and at step 430, the controller verifies/checks criteria of histogram, as depicted in FIG. 4B. At step 435 the controller verifies if the histogram has reached a predefined value indicating that the medical imaging endoscope of the imaging system is placed within a patient body. It is noted that the histogram verification conducted by the controller is preformed per received histogram. In one embodiment, the controller verifies the position of the distal tip of the medical imaging endoscope as “IN” the body or “OUT” of the body by measuring and comparing the normalized histogram with a predefined value set. For example, in case a maximum normalized color value of the red channel and the maximum intensity level of the red channel exceed at least one specific predefined threshold, the system can detect the medical imaging endoscope device as “IN” the body or “OUT” of the body. In case the distal tip position is detected to be “OUT” the patient body, the system can progress to step 440 to operate “OUT” actions of a conditional event set. For example, the system can stop the LED's activities, in case the distal tip position is detected to be “OUT” of the patient body. In some cases, such actions can be declaring an “end of a medical procedure” to an external system such as a reporting system. For example, a reporting system used by the facility wherein the medical procedure is performed. In some cases, upon completing the actions according to the conditional event set the system can return to step 420 to acquire a new image frame.

In some cases, wherein the distal tip position is detected to be “IN” the patient body the system can proceed to step 450. In such cases, the system can perform an operational event according to a predefined conditional event set. In some cases, performing such an operational event, may be conducted by the controller. In some cases, such actions can be start or stop recording the digital video received by the camera/s, start or stop LEDs activity in the distal tip, start or stop insufflation process, and the like. In some cases, such actions may be declaring a “start of a medical procedure” to the external system such as the reporting system. For example, the reporting system used by the facility wherein the medical procedure is performed. In some cases, upon completing the actions according to the conditional event set the system can return to step 420 to acquire another image frame. In some other cases, step 450 is an end step of the process, and upon proceeding to step 450, e.g., identifying the distal tip of the medical imaging endoscope position to be “IN” the patient body, the medical imaging system maintains the conditional event set.

FIG. 4B discloses a method of managing the decision steps of a classifier comparing criteria of color histogram, in accordance with step 430 of FIG. 4A. At step 505 a medical imaging system comprises a controller (such as controller 255 of FIG. 2B) and an image classifier adapted to calculate the histogram of RGB color of a frame/image and or video signal received at the at least one camera of a distal tip (such as distal tip section 250 of FIGS. 2A-2B) of the medical imaging system. The histogram of RGB color calculated by the classifier may define received color values and received intensity levels therein. In some cases, the color value and intensity level may be determined by separating the red, green, and blue channels/colors and measuring the color value and intensity level of the separated colors. In some other case, the system can utilize histogram representing the number of pixels in each color. Next, at step 510 the image classifier of the system normalizes the RGB colors of the histogram to match maximum predefined color, as aforementioned. Thereafter at step 515 the image classifier identifies the maximum color value of each of the RGB channels. In such cases, the image classifier can identify the maximum normalized color value of the red, the maximum normalized color value of the green, and the maximum normalized color value of the blue. In some cases, wherein the color histogram is utilized, the maximum number of pixels in each color can be measured.

At step 520 the system determines if the maximum color value of the red channel has exceeded above the predefined value. In one embodiment, the controller compares the maximum normalized color value of the red channel with the predefined value. If the maximum normalized color value of the red exceeds a predefined value, the controller may progress to step 525. In some embodiments of the present invention, the system can be configured such that, in case the maximum normalized color value of the red has not exceeded a predefined value, the method disclosed herein can progress to step 540 and the rigid scope position can be detected to be “OUT” a patient body. In combination, a new cycle begins again at step 505.

At step 525 the system determines if the maximum color value of the green channel has exceeded above the maximum normalized color value of the blue channel. In one embodiment, the controller compares and verifies if the maximum normalized color value of the green channel is greater than the maximum normalized color value of the blue channel. If the maximum normalized color value of the green channel is greater than maximum normalized color value of the blue channel, the image classifier progresses to step 530 of the method disclosed herein. If the maximum normalized color value of the green has not exceeded than maximum normalized color value of the blue, the method disclosed herein may progress to step 540 and the rigid scope position can be detected to be “OUT” the patient body. In combination, a new cycle begins again at step 505.

At step 530 the system determines if the maximum color value of the red channel has exceeded above the maximum normalized color value of the green channel. In one embodiment, the image controller compares and verifies if the maximum normalized color value of the red channel is greater than maximum normalized color value of the green channel. If the maximum normalized color value of the red is greater than maximum normalized color value of the green, the classifier may progress to the step 535 of the method disclosed herein. If the maximum normalized color value of the red has not exceeded the maximum normalized color value of the green, the method disclosed herein may progress to step 540 and the rigid scope position may be detected to be “OUT” the patient body. In combination, a new cycle begins again at step 505.

At step 535 the image controller calculates the maximum intensity level of the red channel. Next, at step 545 the system determines if the maximum intensity level of the red channel has exceeded above a predefined maximum intensity value. In one embodiment, the classifier compares the maximum intensity level of the red channel with the predefined value. If the maximum intensity level of the red exceeds a predefined value, the classifier may progress to step 550. In some embodiments of the present invention, the system can be configured such that, in case the maximum intensity level of the red has not exceeded a predefined value, the method disclosed herein may progress to step 540 and the rigid scope position can be detected to be “OUT” a patient body. In combination, a new cycle begins again at step 505.

At step 545 the system can conclude the results accumulated in in the steps of the method, as disclosed aforementioned, and then, the system decides whether the rigid scope is within the patient body, or out of the patient body. In some cases, wherein the system concludes that the rigid scope is “IN” the patient body, the system may progress to step 550 to perform the conditional events associated with the status of rigid scope “IN” the patient body. Such events may be start recording with at least one of the cameras of the rigid scope, turn on the LED's or a LED in the rigid scope, and the like. In some cases, such events may be declaring a “start of a medical procedure” to the external system such as the reporting system. For example, the reporting system used by the facility wherein the medical procedure is performed.

In some cases, wherein the system concludes that the rigid scope is “OUT” of the patient body, the system may progress to step 540 to perform the conditional events associated with the status of rigid scope “OUT” the patient body. The conditional events may be such as, to stop recording with at least one of the cameras of the rigid scope, turn of the LED's or a LED in the rigid scope, and the like. In some cases, such events may be declaring an “end of a medical procedure” to an external system such as a reporting system. For example, a reporting system used by the facility wherein the medical procedure is performed.

In some embodiments of the present invention, the usage of RGB color range can be replaced with YCbCr color values or other scales which can be utilized by a person having ordinary skills in the art to measure the intensity value of the color.

While the disclosure has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A method operable by a controller interlocking with a medical imaging endoscope device, comprising:
detecting by the controller a video signal resulted by an image captured by an optical gear of the medical imaging endoscope device;
receiving, by the controller, the histograms representing the intensity levels of colors of the color space of said image;
conducting a comparison process by the controller to compare the received color values with at least one predefined threshold;
determining whether a distal tip of the medical imaging endoscope device position is within a body of a patient, according to the results of the comparison process, and
initiating operational events according to a predefined conditional event set, in case the rigid scope position is detected to be inside the body of the patient.

2. The method of claim 1, further comprises initiating operational events according to a predefined conditional event set, in case the distal tip position is detected to be outside of the body of the patient.

3. The method of claim 1, wherein said histograms is an RGB color value.

4. The method of claim 1, wherein said histograms is a YCbCr color value.

5. The method of claim 1, wherein the histograms are received from a classifier interlocking with the controller.

6. The method of claim 1, wherein the controller is configured to receive the video signal and identify the histograms representing the intensity levels of histograms of the color space of said image.

7. The method of claim 1, further comprises declaring a start of medical procedure to an external system.

8. The method of claim 1, further comprises declaring an end of medical procedure to an external system.

9. A medical imaging system for detecting the position of a medical imaging endoscope, the system, comprising:
an optical gear adapted to capture a video frame/image and or a video signal, wherein the optical gear is located at a distal tip of said medical imaging endoscope device;
a controller for analyzing a histogram representing an intensity level of a colored light received at the medical imaging endoscope, wherein said controller compares said intensity level to specific thresholds determine the position of said medical imaging endoscope, wherein said system is configured to perform the following steps:
detecting by the controller a video signal resulted by an image captured by said optical gear of the medical imaging endoscope device;
receiving, by the controller, the histogram representing the intensity levels of histogram of the color space of said image;
conducting a comparison process by the controller to compare the received histograms with at least one predefined threshold;
determining whether a distal tip of the medical imaging endoscope device position is within a body of a patient, according to the results of the comparison process;
initiating operational events according to a predefined conditional event set, in case the rigid scope position is detected to be inside the body of the patient.

10. The medical imaging system of claim 9, further comprises initiating operational events according to a predefined conditional event set, in case the distal tip position is detected to be out of the body of the patient.

11. The medical imaging system according to claim 9, wherein said histogram is of RGB color values.

12. The medical imaging system according to claim 9, wherein said histogram is of YCbCr color values.

13. The medical imaging system according to claim 9, wherein the controller further initiates an operational event once said intensity level meets said specific thresholds.

14. The medical imaging system according to claim 9, wherein the controller interlocks with an image classifier, wherein said image classifier is adapted to detect and manipulate an RGB image color histogram received form an image processing module.

15. The controller according to claim 13, wherein said operational event is start or stop recording a digital video received by the optical gear.

16. The controller according to claim 13, wherein said operational event is start or stop illumination activity of an illuminator in the distal tip, wherein said illuminator is integrated within said optical gear.

17. The controller according to claim 13, wherein said operational event is start or stop an insufflation process.

18. The controller according to claim 14, wherein said image classifier further configured to define parameters such as color values and intensity levels of each image color within the received video frame/image and or a video signal.

19. A medical imaging system for detecting the position of a medical imaging endoscope, the system comprising:
an optical gear adapted to capture a video frame/image and or a video signal and located at a distal tip of said medical imaging endoscope device;
a controller for analyzing an intensity level of a colored light received at the medical imaging endoscope, wherein said controller compares said intensity level to specific thresholds determine the position of said medical imaging endoscope.
